(19) United States
(12) Patent Application Publication
(10) Pub. No.: US 2022/0330529 A1
(43) Pub. Date: Oct. 20, 2022

(54) METHODS FOR GENERATING ANIMAL MODELS FOR NONALCOHOLIC FATTY LIVER DISEASE

(71) Applicant: INNOLAND BIOSCIENCES (ZHEJIANG) CO., LTD., Hangzhou,Zhejiang (CN)

(72) Inventors: Guodong ZHANG, New Iberia, LA (US); Xiaoli WANG, Taicang, Jiangsu (CN); Yixin WANG, Taicang, Jiangsu (CN)

(21) Appl. No.: 17/639,923

(22) PCT Filed: Sep. 3, 2020
(86) PCT No.: PCT/CN2020/113199

§ 371 Date: Mar. 3, 2022

Related U.S. Application Data
(60) Provisional application No. 62/894,974, filed on Sep. 3, 2019.
(60) Provisional application No. 63/031,575, filed on May 29, 2020.

(51) Int. Cl. A01K 67/02; A61K 31/02

(52) U.S. Cl. CPC A01K 67/02; A61K 31/02; A01K 2227/105; A01K 2267/03

(57) ABSTRACT

Non-human animal models of non-alcoholic fatty liver disease (NAFLD) are provided. Compositions and methods for producing the non-human animal models and uses of the non-human animal models to screen and evaluate agents for treating or preventing NAFLD are also provided.

### CROSS-REFERENCE TO RELATED APPLICATIONS

The present application claims the benefit of US Provisional Application Nos 62/894,974, filed Sep. 3, 2019, and 63/031,575, filed May 29, 2020, both of which are incorporated herein by reference in their entirety.

### FIELD OF THE INVENTION

The present invention generally relates to animal models, the compositions and methods making the same and the uses thereof. In particular, the present invention relates to methods for generating animal models for nonalcoholic fatty liver disease.

### BACKGROUND

Non-alcoholic fatty liver disease (NAFLD) is a condition in which excess fat is stored in the liver of a person without excessive alcohol consumption. It is estimated that 25% of the world's general population meet the criteria for a diagnosis of NAFLD; NAFLD is more common in men and increases with age. The incidence of NAFLD also appears to be stratified across ethnic groups in the order of Hispanics (45%), Caucasians (33%) and African-Americans (24%).

The initial stage of NAFLD is characterized by the accumulation of ectopic fat in hepatocytes, i.e. steatosis. Steatosis is generally a benign, asymptomatic condition; however, with concurrent obesity/metabolic disturbances, steatosis can progress to non-alcoholic steatohepatitis (NASH) and in severe cases hepatocellular carcinoma (HCC) and liver failure. Histologically NASH is characterized by hepatocellular ballooning, inflammation and increased risk for liver fibrosis. Unlike benign steatosis, NASH represents a significant health threat that progresses to fibrosis/cirrhosis in 10-28% of patients. Further progression from NASH to fibrosis/cirrhosis is highly predictive of mortality in these patients.

The study of human NAFLD and its progression is hampered by the slow development of disease, which may take decades, as well as lack of tools available for staging the disease. The significant health threat ascribed to NASH versus the often-benign steatosis, makes early differentiation a necessary step in predicting which patients will progress to fibrosis and eventually liver failure. Currently, the staging of the fatty liver environment relies on histological evaluation from liver biopsy which is invasive, expensive and not practical for screening all NAFLD patients. While much research is ongoing to identify non-invasive tools for staging, biopsy remains the gold standard and reliable clinical biomarkers are not yet available. Thus, attempts have been made to develop rodent models of fatty liver disease to aid in the investigation of the pathophysiological and morphological findings characteristic of NAFLD, as well as histological characteristics such as steatosis, interlobular inflammation, hepatocellular ballooning, fibrosis and be susceptible to liver tumors seen in humans.

Over the last several years, investigators have taken different approaches to developing mouse models of NAFLD and NASH, including methionine-choline deficient diet (Machado MV et al. PLoS One (2015) 10(5):e0127991), high fat diets with and without fructose in C57BL/6J and ob/ob mice (Charlton M et al. Am J Physiol Gastrointest Liver Physiol (2011) 301(5):G825-34; Itagaki H et al. Int J Clin Exp Pathol (2013) 6(12):2683-96; Kristiansen MN et al. World J Hepatol (2016) 8(16):673-84; Tetri LH et al. Am J Physiol Gastrointest Liver Physiol (2008) 295(5):G987-95) and the STAM model where 4 day old mice are given streptozotocin plus high fat diet (Jojima T et al. Diabetol Metab Syndr.8:45; Saito K et al. Sci Rep (2016) 5:12466). Carbon tetrachloride (CC14) has been used to induce liver fibrosis in mice model.

However, these animal models fail to accurately display the characteristic of NAFLD. For example, initial attention has been placed on producing fibrosis as quickly as possible with the methionine-choline deficient (MCD) diet. The mice on the MCD diet are not obese, actually loose significant body weight (30%), and are not insulin resistant or hyperlipidemic during disease progression. The STAM model is characterized by type 1 diabetes induced with streptozotocin, rather than type 2 diabetes on a high fat diet and produces fibrosis after 12 weeks on diet and eventually HCC. For CCl₄—induced fibrosis model, the high chemical dosage (0.8-1.0 ml/kg) required to generate the liver fibrosis phenotype causes severe body weight loss, which resulted in lacking dysmetabolic phenotype, such as obesity and hyperinsulinemia.

The inventors of this application have developed a NAFLD/NASH model using MS-NASH (metabolic syndrome NASH) mouse fed with high-fat high-fructose diet (see U.S. patent application Ser. No. 16/013,953, the entire disclosure of which is incorporated herein through reference). However, it takes 20 weeks of high-fat high-fructose diet feeding to reach mild to moderate fibrosis stage. Therefore, there is a continuing need to develop new animal models for NAFLD/NASH.

### SUMMARY OF INVENTION

In one aspect, the present disclosure provides a method for producing a non-human animal model of non-alcoholic fatty liver disease (NAFLD). In an embodiment, the method comprising obtaining a MS-NASH mouse at a young age and feeding the MS-NASH mouse with a diet of high-fat, high cholesterol and high fructose and administering CCl₄ to the MS-NASH mouse for a period of time.

In certain embodiments, the CCl₄ is administered at about 0.05-0.2 ml per kg body weight of the MS-NASH mouse. In certain embodiments, the CCl₄ is administered via intraperitoneal injection. In certain embodiments, the CCl₄ is administered twice or three times a week.

In certain embodiments, the diet comprises 40% kcal fat and 20% kcal fructose. In certain embodiments, the diet comprises 40% kcal fat and 5% fructose in drinking water.

In certain embodiments, the NAFLD is steatosis, non-alcoholic steatohepatitis (NASH), cirrhosis or liver cancer.

In certain embodiments, the young age is about 3-8-week old (e.g., 3, 4, 5, 6, 7, or 8 weeks old).

In certain embodiments, the period of time is about 4 weeks, 8 weeks, 12 weeks, 16 weeks or 20 weeks.

In a second aspect, the present disclosure provides a non-human animal model of NAFLD. In certain embodiments, the non-human animal model of NAFLD is produced by feeding a MS-NASH mouse of a young age with a diet of high-fat, high cholesterol and high fructose and administering to the MS-NASH mouse CCl₄ for a period of time.

In a third aspect, the present disclosure provides a method of screening for an agent for treating or preventing NAFLD. In one embodiment, the method comprises: (a) administering a candidate agent to the non-human animal model described herein; and (b) evaluating an ameliorative effect on the NAFLD.

In a fourth aspect, the present disclosure provides a method of evaluating a medicament for treating NAFLD. In one embodiment, the method comprises: (a) administering the medicament to the non-human animal model described herein; and (b) evaluating an ameliorative effect on the NAFLD.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present invention.

FIGS. 1A and 1B show the growth curve of MS-NASH (previously known as FATZO) mouse and comparison with other mouse strains in terms of body weight (FIG. 1A), and body fat % (FIG. 1B).

FIGS. 2A-2D show the NAFLD progression by feeding 40% kcal fat content and 5% fructose in drinking water (WDF) on MS-NASH mice starting at 8 weeks old comparing with normal chow (CD) in terms of body weight (FIG. 2A), body fat % (FIG. 2B), total cholesterol (FIG. 2C), and triglycerides (FIG. 2D).

FIGS. 3A-3D show the liver injury markers progression by feeding 40% kCal fat content and 5% fructose in drinking water (WDF) on MS-NASH mice starting at 8 weeks old comparing with normal chow (CD) in terms of ALT (FIG. 3A), AST (FIG. 3B), liver weight % (FIG. 3C), and liver triglycerides (FIG. 3D).

FIG. 4 shows the histology images of NAFLD progression by feeding 40% kcal fat content and 5% fructose in drinking water (WDF) on MS-NASH mice starting at 8 weeks old comparing with normal chow (CD) as illustrated in gross picture, H&E staining and picrosirius red staining.

FIGS. 5A-5E show the NAS score and fibrosis score of histology findings by feeding 40% kcal fat content and 5% fructose in drinking water (WDF) on MS-NASH mice starting at 8 weeks old comparing with normal chow (CD). FIG. 5A, Steatosis score; FIG. 5B, Ballooning score; FIG. 5C, Lobular inflammation score; FIG. 5D, Fibrosis score; and FIG. 5E, NAFLD activity score.

FIGS. 6A-6C show the NAFLD progression by feeding 40% kcal fat content and 20% kcal fructose content diet (AMLN) on MS-NASH mice starting at 8 weeks old comparing with normal chow (CD) and WDF diet in terms of body weight (FIG. 6A), total cholesterol (FIG. 6B), and triglycerides (FIG. 6C)

FIGS. 7A-7D show the liver injury markers progression by feeding 40% kcal fat content and 20% kcal fructose content diet (AMLN) on MS-NASH mice starting at 8 weeks old comparing with normal chow (CD) and WDF diet in terms of ALT (FIG. 7A), AST (FIG. 7B), liver weight % (FIG. 7C), and liver triglycerides (FIG. 7D).

FIGS. 8A-8F show effects of high dose CCL₄ (0.2 mL/kg) twice weekly for 3 weeks in MS-NASH mice fed control diet (CD) or Western diet supplemented with fructose (WDF). FIGS. 8A-8E show body weight (FIG. 8A), daily food (FIG. 8B), calories intake (FIG. 8C), serum ALT (FIG. 8D) and AST (FIG. 8E) before and after repeated high dose CCl₄. FIG. 8F shows time course of acute response of ALT and AST to a single high dose CCl₄ in mice on control diet (CD). Data represent as mean±SEM.

FIGS. 9A-9H show histopathology in MS-NASH mice fed control diet (CD) or Western diet supplemented with fructose (WDF) treated high dose CCl₄ (0.2 mL/kg) twice weekly for 3 weeks. FIGS. 9A-9F show representative images of H&E (Hematoxylin and Eosin) and PSR (Picro Sirius Rd) staining in animals fed CD (FIGS. 9A and 9B); and WDF treated without (FIGS. 9C and 9D) or with (FIGS. 9E and 9F) CCl₄, respectively. Arrows in FIG. 9F indicate fibrosis. FIG. 9G shows pathology scores of steatoses (0-3), lobular inflammation (0-3), ballooning (0-2), NAFLD activity (0-8). FIG. 9H shows fibrosis score (0-4) and percentage fibrosis area, quantitatively analyzed as total PSR positive staining area over total liver section area scanned and processed by HALO software. Data represent as mean±SEM. # p<0.05, ### p<0.005 comparing with CD group; * p<0.05, *** p<0.005 comparing with WDF group by one-way ANOVA analysis.

FIGS. 10A-10E show effects of low dose CCl₄ (0.08 mL/kg) twice weekly for 8 weeks in MS-NASH mice fed Western diet supplemented with fructose (WDF). FIGS. 10A-10C show body weights (FIG. 10A), serum ALT (FIG. 10B) and AST (FIG. 10C) levels before and after low dose CCl₄. In-life AST and AST levels at weeks 11, 12 and 14 were measured—72 hours; and the terminal one at week 16 measured—24 hours, after CCl₄ administration. FIGS. 10D-10F show liver weight (FIG. 10D), cholesterol (FIG. 10E), and triglycerides (FIG. 10F) measured at the end of the study. Data represent as mean±SEM. * p<0.05, *** p<0.005, WDF vs. WDF+CCl₄ group by Holm-Sidak t-test.

FIGS. 11A-11F show histopathology in Western diet supplemented with fructose (WDF) fed MS-NASH mice treated with low dose CCl₄ (0.08 mL/kg) for 8 weeks. FIGS. 11A-11D show representative images of H&E and PSR staining in animals without (FIGS. 11A and 11B) or with (FIGS. 11C and 11D) CCl₄. Heavy arrows in FIGS. 11A and 11C indicate macrovesicular vacuolation steatosis and light arrows indicate microvesicular ballooning; and arrow in FIG. 11D indicate fibrosis. FIG. 11E shows pathology scores of steatoses (0-3), lobular inflammation (0-3), ballooning (0-2), and NAFLD activity (0-8), and fibrosis (0-4). FIG. 11F shows quantitative histology analyzed as percentage of steatosis and fibrosis area, and cell counts of inflammation and hepatic ballooning by Reveal ImageDx software. Data represented as mean±SEM. *** p<0.005, WDF vs. WDF+CCl₄ group using Holm-Sidak t-test.

FIGS. 12A-12F show therapeutic effects of obeticholic acid (OCA, 30 mg/kg, QD (once daily)) in Western diet supplemented with fructose (WDF) fed MS-NASH or C57B1/6 mice treated low dose CCl₄ (0.08 mL/kg) twice weekly for 8 weeks. FIGS. 12A-12C show body weight (FIG. 12A), serum ALT (FIG. 12B) and AST (FIG. 12C). FIGS. 12D-12F show terminal liver weight (FIG. 12D), triglycerides (FIG. 12E) and cholesterol (FIG. 5F). Data represented as mean±SEM. * p<0.05, *** p<0.005, Veh. vs OCA groups by Holm-Sidak t-test.

FIGS. 13A-13F show histopathology of obeticholic acid (OCA, 30 mg/kg, QD) treatment on Western diet supplemented with fructose (WDF) fed MS-NASH or C57B1/6 mice under low dose CCl₄ (0.08 mL/kg) twice weekly for 8 weeks. FIGS. 13A-13H show representative images of H&E and PSR staining in MS-NASH mice on WDF treated with vehicle (FIGS. 13A and 13B) or OCA (FIGS. 13C and 13D) or C57B1/6 mice with vehicle (FIGS. 13E and 13F) or OCA (FIGS. 13G and 13H). Heavy arrows in FIGS. 13A and 13E indicate steatosis, light arrow in FIG. 13A indicates microvesicular ballooning, and arrows in FIGS. 13B and 13F indicate fibrosis. FIG. 13J shows pathologist scores of steatosis (0-3), lobular inflammation (0-3), ballooning (0-2) and NAFLD Activity (0-8), as well as fibrosis (0-4). FIG. 13K shows quantitative imaging analysis of steatosis, inflammatory cell infiltration, hepatic ballooning and fibrosis by Reveal ImageDx software. Data represented as mean±SEM. * p<0.05, *** p<0.005, Veh VS OCA group using Holm-Sidak t-test.

FIG. 14 shows comparison of survival rates in Western diet supplemented with fructose (WDF) fed MS-NASH or C57B1/6 mice under CCl₄ twice weekly.

FIGS. 15A-15D show correlation between Pathology scores and Reveal ImageDx analysis. FIGS. 15A-15D show correlations between pathology scores for steatosis (FIG. 15A), lobular inflammation (FIG. 15B), hepatocyte ballooning (FIG. 15C), and fibrosis (FIG. 15D) and Reveal ImageDx quantification by simple linear correlation with Pearson's coefficients. All the Pearson's correlation coefficient r values are statistically significant.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

The following definitions are provided to assist the reader. Unless otherwise defined, all terms of art, notations and other scientific or medical terms or terminology used herein are intended to have the meanings commonly understood by those of skill in the art. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, an “animal model” refers to a living organism with an inherited, naturally acquired, or induced pathological process that in one or more respects resembles the same phenomenon in a person.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

As used herein, MS-NASH mouse refers a polygenic model developed by cross-breeding C57BL/6J mice with AKR/J mice and then selectively in-breeding for obesity, hyperglycemia and insulin resistance for at least 30 generations to genetic homogeneity (see U.S. patent application Ser. No. 16/013,953, the entire disclosure of which is incorporated herein through reference). This model is unique in that it possesses an intact leptin pathway, unlike the ob/ob or db/db mouse monogenic models of obesity and type 2 diabetes, thereby making it more translatable to the human disease.

As used herein, obeticholic acid (OCA) refers to a semi-synthetic bile acid that acts on the nuclear farnesoid X receptor (FXR) which is expressed predominantly in liver, kidney and intestine to regulate bile acid homeostasis, hepatic lipid metabolism as well as immune function. It was originally developed for the treatment of primary biliary cholangitis and is currently being tested for NASH in several clinical trials. OCA has shown effects of improvement in liver function and pathology in human and pre-clinical NASH models.

Animal Models of NAFLD

Non-alcoholic fatty liver disease (NAFLD) is an all-encompassing term used to describe the fatty liver environment in the absence of excessive alcohol consumption. It is estimated that 25% of the world's general population meet the criteria for a diagnosis of NAFLD; NAFLD is more common in men and increases with age. The incidence of NAFLD also appears to be stratified across ethnic groups: Hispanics (45%)>Caucasians (33%)>African-Americans (24%).

The initial stage of NAFLD is characterized by the accumulation of ectopic fat in hepatocytes (steatosis). Steatosis is generally a benign, asymptomatic condition; however, with concurrent obesity/metabolic disturbances, steatosis can progress to non-alcoholic steatohepatitis (NASH) and in severe cases hepatocellular carcinoma (HCC) and liver failure. Histologically NASH is characterized by hepatocellular ballooning, inflammation and increased risk for liver fibrosis. Unlike benign steatosis, NASH represents a significant health threat that progresses to fibrosis/cirrhosis in 10-28% of patients. Further progression from NASH to fibrosis/cirrhosis is highly predictive of mortality in these patients.

The study of human NAFLD and its progression is hampered by the slow (decades) development of disease as well as tools available for staging the disease. Therefore, an animal model accurately displays the characteristics of NAFLD is needed.

Therefore, the present disclosure in one aspect provides a method for producing a non-human animal model of non-alcoholic fatty liver disease (NAFLD). In an embodiment, the method comprising obtaining a MS-NASH mouse at a young age and feeding the MS-NASH mouse with a diet of high-fat, high cholesterol and high fructose and administering CCl₄ to the MS-NASH for a period of time.

As used herein, a mouse is considered young from about 3 weeks to about 8 weeks old. In some embodiments, the young age as described herein is about 3 weeks, 4 weeks, 5 weeks, 6 weeks, 7 weeks or 8 weeks old.

As used herein, mouse diet refers to the sum of food consumed by a mouse, especially a mouse raised in a laboratory or facility. The ingredients and compositions of mouse diet are known in the art. For example, ingredients of a formulated lab mouse diet may include, without limitation, ground corn, ehulled soybean meal, whole wheat, fish meal, wheat middlings, porcine animal fat preserved with BHA and citric acid, cane molasses, porcine meat and bone meal, ground oats, wheat germ, brewers dried yeast, dehydrated alfalfa meal, dried beet pulp, whey, calcium carbonate, salt, menadione dimethylpyrimidinol bisulfite (source of vitamin K), choline chloride, cholecalciferol, DL-methionine, vitamin A acetate, pyridoxine hydrochloride, dl-alpha tocopheryl acetate (form of vitamin E), folic acid, thiamine mononitrate, nicotinic acid, calcium pantothenate, riboflavin supplement, vitamin B 12 supplement, manganous oxide, zinc oxide, ferrous carbonate, copper sulfate, zinc sulfate, calcium iodate, cobalt carbonate.

As used herein, a mouse diet of high-fat means a diet in which about 20-40% kcals (e.g., about 20%, 25%, 30%, 35%, 40%) are from fat.

In one example, a high-fat mouse diet has the formulation as listed in Table 1.

TABLE 1
formulation of high-fat (40% kcal) mouse diet

| Class description | Ingredient | Grams | | kcal |
|---|---|---|---|---|
| Protein | Casein, Lactic, 30 Mesh | 195.0 | g | 780 |
| Protein | Methionine, DL | 3.0 | g | 12 |
| Carbohydrate | Sucrose, Fine Granulated | 350.0 | g | 1355 |
| Carbohydrate | Lodex 10 | 100.0 | g | 0 |
| Carbohydrate | Starch, Corn | 50.0 | g | 200 |
| Fiber | Solka Floc, FCC200 | 50.0 | g | 0 |
| Fat | Butter, Anhydrous | 200.0 | g | 1434 |
| Fat | Corn Oil | 10.0 | g | 90 |
| Mineral | S10001A | 17.5 | g | 0 |
| Mineral | Calcium Phosphate, Dibasic | 17.5 | g | 0 |
| Mineral | Calcium Carbonate, Light, USP | 4.0 | g | 0 |
| Vitamin | Choline Bitartrate | 2.0 | g | 0 |
| Vitamin | V10001C | 1.0 | g | 4 |
| Anti-oxidents | Ethoxyquin | 0.0 | g | 0 |
| Special | Cholesterol, NF | 1.5 | g | 0 |
| | Total: | 1001.5 | g | 3875 |

As used herein, a mouse diet of high-fructose means a diet which contains about 5-20% (e.g., about 5%, 10%, 15% or 20%) fructose, e.g., 5% fructose in drinking water.

In certain embodiments, the diet comprises fat of 40% kcal and 5% fructose in drinking water.

In certain embodiments, the diet comprises 40% kcal fat and 20% kcal fructose. In one example, a high-fat high-fructose mouse diet has the formulation as listed in Table 2.

TABLE 2
formulation of high-fat (40% kcal)
high-fructose (20% kcal) mouse diet

| Class description | Ingredient | Grams | | kcal |
|---|---|---|---|---|
| Protein | Casein | 200 | g | 800 |
| Protein | L-Cystine | 3 | g | 12 |
| Carbohydrate | Maltodextrin 10 | 100 | g | 400 |
| Carbohydrate | Fructose | 200 | g | 800 |
| Carbohydrate | Sucrose | 96 | g | 384 |
| Fiber | Cellulose | 50 | g | 0 |
| Fat | Soybean Oil | 25 | g | 225 |
| Fat | Lard | 20 | g | 180 |
| Fat | Palm Oil | 135 | | 1215 |
| Mineral | Mix S10026 | 10 | g | 0 |
| Mineral | DiCalcium Phosphate | 13 | g | 0 |
| Mineral | Calcium Carbonate | 5.5 | g | 0 |
| Mineral | Potassium Citrate, 1H₂O | 16.5 | | 0 |
| Vitamin | Choline Bitartrate | 2 | g | 0 |
| Vitamin | Mix V10001 | 10 | g | 40 |
| Special | Cholesterol | 18 | | 0 |
| | Total: | 904 | g | 4056 |

As used herein, feeding a mouse with a diet means the mouse is fed mainly with the diet, i.e., at least 80%, 85%, 90% of the food fed to the mouse is based on the diet.

In certain embodiments, the period of time is about 2 weeks, 3 weeks, 4 weeks, 5 weeks, 6 weeks, 7 weeks, 8 weeks, 9 weeks, 10 weeks, 12 weeks, 14 weeks, 16 weeks, 18 weeks, 20 weeks or more.

In certain embodiments, while the MS-NASH mouse is fed with high-fat and high-fructose diet, the mouse is administered with carbon tetrachloride (CCl₄).

In certain embodiments, CCl₄ is administered at an amount of 0.05-0.2 mg per kg body weight of the MS-NASH mouse. In certain embodiments, the CCl₄ is administered at about 0.05, 0.06, 0.07, 0.08, 0.09, 0.1, 0.11, 0.12, 0.13, 0.14, 0.15, 0.16, 0.17, 0.18, 0.19 or 0.2 ml per kg body weight of the MS-NASH mouse.

The CCl₄ may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes. In certain embodiments, the CCl₄ is administered via intraperitoneal injection.

The CCl₄ may be administered at any frequency that results in the development of NAFLD. In certain embodiment, the CCl₄ is administered once, twice or three times a week.

Feeding MS-NASH mice with high-fat high-fructose diet causes the MS-NASH mice to develop NAFLD. In one example, MS-NASH mice fed the diet comprising 40% kcal fat and 5% fructose in drinking water (WDF diet) develop NAFLD and NASH with progressive steatosis and fibrosis with consistent ballooning and inflammation when compared to MS-NASH mice fed regular chow diet (CD). On gross necropsy, the livers from the mice fed with the WDF diet are significantly larger and pale in color when compared to mice fed CD. In the plasma, increases in the liver enzymes, ALT (alanine aminotransferase) and AST (aspartate aminotransferase), and cholesterol are observed in the WDF diet fed animals and remained significantly higher compared to values obtained from animals on CD. Plasma triglycerides are not elevated in the WDF diet fed animals when compared to the CD fed animals; as observed in the ob/ob NASH models. However, liver triglycerides are elevated in mice fed the WDF diet compared to mice fed CD. The mice fed with the WDF diet have elevated glucose levels but do not become diabetic as compared to the mice fed CD; a common finding seen in the high fat/fructose fed C57BL/6 and ob/ob models.

On gross necropsy, the livers from WDF diet fed MS-NASH mice are pale in color and had significantly higher liver/% BW ratios when compared to their CD fed groups. Histologically, the livers from the MS-NASH mouse fed the WDF diet, demonstrate steatosis on diet which progress to steatohepatitis characterized by balloon degeneration, lobular inflammation and fibrosis. The composite NAS (NAFLD activity) score in the MS-NASH mouse fed WDF indicates “definitive” NASH. Mild fibrosis is observed in MS-NASH mice fed with the WDF diet and progressed to moderate fibrosis in about 20 weeks.

Carbon tetrachloride (CCl₄) has been used to induce liver fibrosis in mice model. The high chemical dosage (0.8-1.0 ml/kg) required to generate the liver fibrosis phenotype causes severe body weight loss, which resulted in lacking dysmetabolic phenotype, such as obesity and hyperinsulinemia.

Feeding MS-NASH mice with high-fat high-fructose diet along with small amount administration of CCl₄ induces the MS-NASH mice to develop faster and more severe liver fibrosis compared with MS-NSH mice fed with high-fat high-fructose diet only. Further, in contrast to the pure chemically-induced NASH model using CCl₄, the combination of high-fat high-fructose diet and small dose administration of CCl₄ does not reduce the body weight gain and liver steatosis. Therefore, in one aspect of the present invention, small dose of CCl₄ is utilized to accelerate liver fibrosis in high fat diet fed spontaneous dysmetabolic moue with intact leptin pathway to generate a NASH model that maintains the dysmetabolic phenotype and also achieves severe liver fibrosis within 12-16 weeks of high fat diet feeding. In one example, MS-NASH mice fed with WDF along with administration of 0.08 ml/kg CCl₄ have similar body weight growth curve as the mice fed with CD. The MS-NASH mice fed with WDF along with administration of 0.08 ml/kg CCl₄ showed accelerated NAFLD progression and fibrosis development.

In a second aspect, the present disclosure provides a non-human animal model of NAFLD produced by the methods described herein. In certain embodiments, the non-human animal model of NAFLD is produced by feeding a MS-NASH mouse of a young age with a diet of high-fat and high fructose and administration of CCl₄ to the MS-NASH mouse for a period of time.

Use of the Animal Models

In another aspect, the present disclosure provides a method of screening for an agent for treating or preventing NAFLD. In one embodiment, the method comprises: (a) administering a candidate agent to the non-human animal model described herein; and (b) evaluating an ameliorative effect on the NAFLD.

In yet another aspect, the present disclosure provides a method of evaluating a medicament for treating NAFLD. In one embodiment, the method comprises: (a) administering the medicament to the non-human animal model described herein; and (b) evaluating an ameliorative effect on the NAFLD.

Multiple drugs have been in the development stage for the specific treatment of NASH. Among them, obeticholic acid (OCA), a semi-synthetic bile acid that acts on the nuclear farnesoid X receptor (FXR) is in the most advanced stage of clinical trial with evidence of significant alleviation of plasma liver ALT and AST levels and mild improvement in steatosis, hepatic ballooning, lobular inflammation and fibrosis. In pre-clinical rodent studies, OCA has shown benefits in reducing hepatic lipid accumulation, liver enzyme activities, steatosis and fibrosis, though the models and dosing regimen selected might largely affect the final manifest of the drug efficacy.

In one example, MS-NASH mice fed with high-fat high fructose diet can be treated with OCA before CCl₄ administration. The OCA treatment before CCl₄ administration significantly improved NAS score, such as steatosis and fibrosis. Therefore, MS-NASH mice fed with high-fat high-fructose diet plus CCl₄ administration can provide the NASH phenotypes in the time frame that is suitable for the anti-NASH drug intervention.

### Example 1

Materials and Methods

Animal Studies

Male MS-NASH (formally FATZO) mice were developed by Crown Bioscience as the new generation of mouse model with obese, metabolic disorder, diabetes and NAFLD/NASH that is more translatable to human diseases. The animals for this study were bred housed individually in IVC cages (Taicang, China) or open ventilated cages (Indianapolis, Ind.), and fed control diet (CD, Purina 5008 chow, LabDiet, St. Louis, Mo.) with distilled water ad libitum for the first 8 weeks after birth, then, stratified into different experimental groups based on body weight, serum ALT and AST. Room temperature was monitored and maintained at 22-26° C. with a 12-hour light cycle (06:00-18:00). C57B1/6J mice (The Jackson Laboratory, Ellsworth, Maine) were used as control strain and housed under the same conditions. All mice were maintained and treated in accordance with the guidelines of Association for Assessment and Accreditation of Laboratory Animal Care (AAALAC), and experimental protocols approved by the Institutional Animal Care and Use Committee (IACUC).

Biochemical Measurements

In all the experiments, body weights were recorded every 4 weeks. At the end of the experiments, all mice were euthanized by CO₂ inhalation and confirmed with cervical dislocation approximately 24 hours after the last CCl₄ administration.

Blood samples: Blood samples during the course of the experiment were collected from the tail or at the end of the experiment from cardiac puncture, from which, serum prepared for measuring AST and ALT by a clinical analyzer (Beckman-Coulter AU480; Brea, Calif.). In the low dose CCl₄ repeated treatment group, in-life blood samples were taken—24 hours, and terminal blood samples taken, —24 hours after CCl₄ doing, respectively. A separate experiment was performed to observe the acute time course at 24, 48 and 72 hours in response to a single dose of CCl₄ at 0.2 mL/kg in MS-NASH mice on CD.

Liver contents: The right lobe of the liver (— 200 mg/animal) was collected and snap frozen in liquid nitrogen, placed in Lysing Matrix D Tubes with distilled water at 20% concentration (MP Biomedicals, Santa Anna, CA), homogenized in a Fastprep-FP120 cell disrupter (Thermo Fisher Savant) in cold condition for 30 seconds. The liver contents of triglyceride and cholesterol were analyzed by a clinical analyzer (Beckman-Coulter AU480) within 30 min of sample preparation.

Histology

Tissue processing: The liver tissues were fixed in 10% neutral buffered formalin (NBF) at 4° C. for 24 hours followed by baths of standard concentrations of alcohol then xylene to prepare the tissues for paraffin embedding. After being embedded in paraffin and cooled, five-micron sections were cut and stained for routine H&E and Picric Sirius Red.

Whole slide digital imaging: The Aperio whole slide digital imaging system was used for imaging. The Aperio Scan Scope CS system was used (360 Park Center Drive, Vista, Calif.). The system imaged all slides at 20×. The scan time ranged from L5 minutes to a maximum time of 2.25 minutes. The whole images were housed and stored in their Spectrum software system and images were shot from the whole slides.

Semi-quantitative scoring by a pathologist: The digital images were evaluated by a well-trained research pathologist blind of different study groups with the standard NASH criteria for semi-quantitative scoring commonly used in preclinical animal models and in patients. Hepatosteatosis, lobular inflammation, and hepatocyte balloon degeneration were scored individually from the H&E staining and then summarized as a standard NAFLD Activity Score (NAS). Fibrosis score was assessed systemically with pattern recognition from PSR staining. Three representative areas per liver were examined and the scores of each parameter from individual animal were averaged.

Computerized quantitative analysis: Computer software with automatic intelligence (AI) machine learning algorithm for histology analysis from Halo (Indica Labs, Albuquerque, N. Mex.) or ImageDx (Reveal Biosciences, San Diego, Calif.) were used to analyze digitally scanned images of H&E and PSR staining for quantitative analysis of steatosis, ballooning, inflammation or fibrosis in a set of the same slides evaluated by the pathologist. The analysis process included automated tissue identification, followed by segmentation of regions of interest for quantification of the following metrics: 1) Steatosis percentage: the area of total lipid accumulation subcategorized micro- or macro-vesicular within the entire section area; 2) Ballooning hepatocyte density: the density of ballooning hepatocytes within the entire section area; 3) Inflammatory cell density: the total number of inflammatory cells within the entire section area. All 3 parameters above were analyzed in the H&E stained section. 4)Fibrosis percentage: the total fibrosis area within the entire section area in the PSR stained section.

Statistics

All values are reported as Mean±SEM, unless noted otherwise. Model characterization were compared in MS-NASH mice on CD or WDF with or without CCl₄; and effects of OCA were compared to vehicle with One-Way ANOVA for multiple groups or Holm-Sidak t-test for 2 groups. Survival curves of total MS-NASH mice and C57B1/6 mice were compared using Log-rank test for trend. Parametric correlation tests were conducted between pathologist scores and ImageDx quantitative analysis using Pearson correlation coefficient r. Statistical differences were denoted as p<0.05 or p<0.005. Prism software (GraphPad, version 8.3) was used for the statistical analysis and graphing.

### Example 2

This example shows that the MS-NASH mouse fed with the WDF diet would generate a model of progressive NAFLD and NASH.

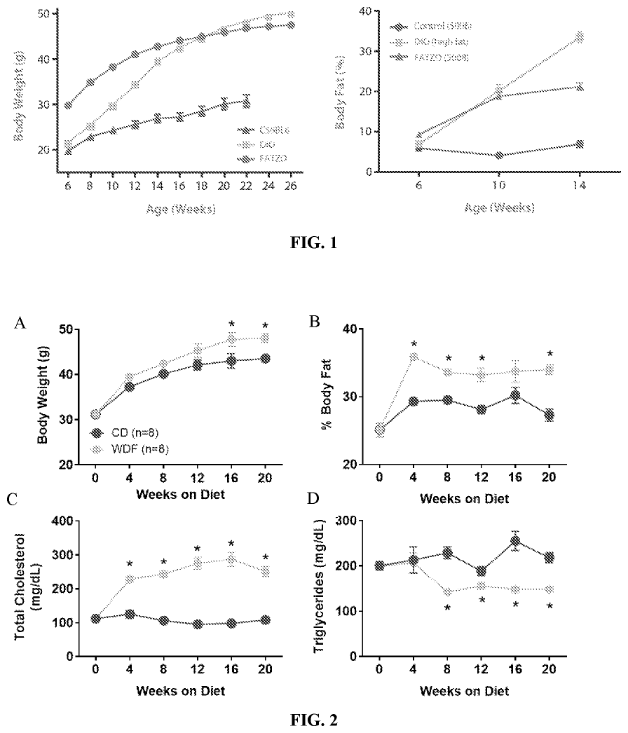

As shown in FIGS. 1A and 1B, MS-NASH mice have increased body weight (FIG. 1A) and body fat (FIG. 1B) compared to wildtype (C56BL6) mice.

As shown in FIGS. 2A-2D, feeding the MS-NASH mice with high-fat (40% kcal fat) and high-fructose (5% fructose in drinking water) diet (WDF diet) exacerbated metabolic disorders, impaired liver function and histological changes assembling to NAFLD/NASH. The MS-NASH mice fed WDF showed a significantly greater increase in body weight (FIG. 2A), associated with a significant increase in body fat compared to the age-matched CD fed mice (FIG. 2B). Blood cholesterol levels were almost 2.5 times higher in WDF group than CD controls after 4 weeks on diet and the levels were consistently higher in WDF group throughout the diet induction period (FIG. 2C), though triglyceride levels were slightly lower (FIG. 2D) in the WDF group.

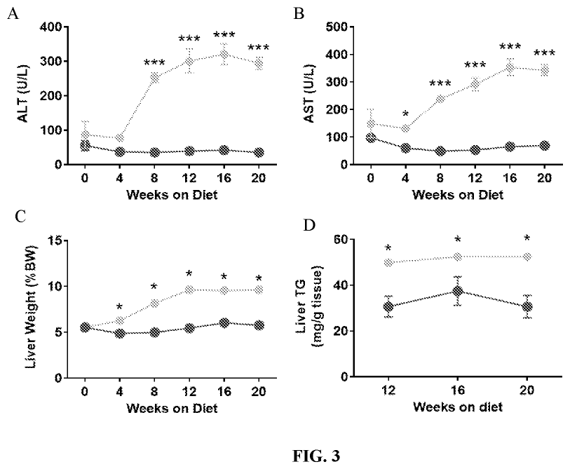

Metabolic stress on the livers of the MS-NASH mice fed with WDF diet caused significant elevation in the liver enzymes, with evidence of almost 6 and 4-fold higher in alanine aminotransferase (ALT) (FIG. 3A) and aspartate transaminase (AST) (FIG. 3B) levels respectively over the 20 weeks of diet exposure compared to that in control diet (CD) fed mice. Liver weight increased over time in both groups, which, however, was significantly higher in the WDF diet group than CD group (FIG. 3C). Liver TG contents measured at weeks 12-20 from mice fed with WDF diet showed 2 folder differences with significantly higher levels compared to that in the mice fed with CD (FIG. 3D).

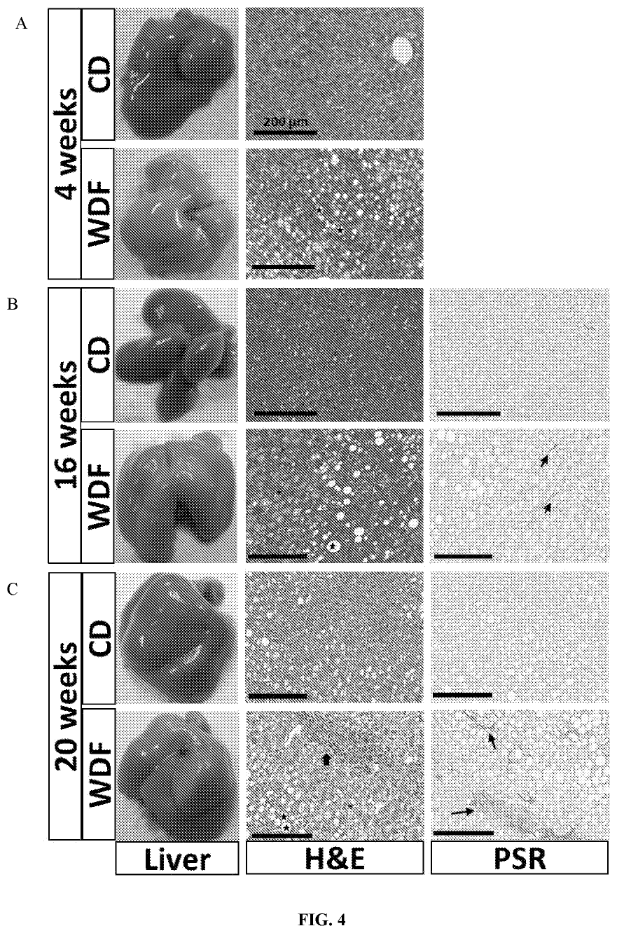

The MS-NASH mice fed with WDF diet developed fatty liver characterized by progressive steatosis, hepatocellular ballooning, lobular inflammation and early stages of fibrosis. During the early progression of NAFLD, the livers from the MS-NASH mice fed with WDF diet were very pale in color upon necropsy compared to that of CD fed mice (FIG. 4). H&E staining demonstrated fully involved steatosis with ballooning as early as 4 weeks on WDF diet group compared to CD group. Over the time, the MS-NASH mice exhibited a progressive worsening of NAFLD. At each time point, the livers of mice fed with WDF diet were paler in color than the corresponding MS-NASH mice fed with CD. Significant histological changes indicative of NAFLD (steatosis, hepatocellular ballooning, lobular inflammation) including mild fibrosis were seen in the liver sections from the group after 16 weeks on WDF diet compared to the corresponding CD fed group (FIG. 4).

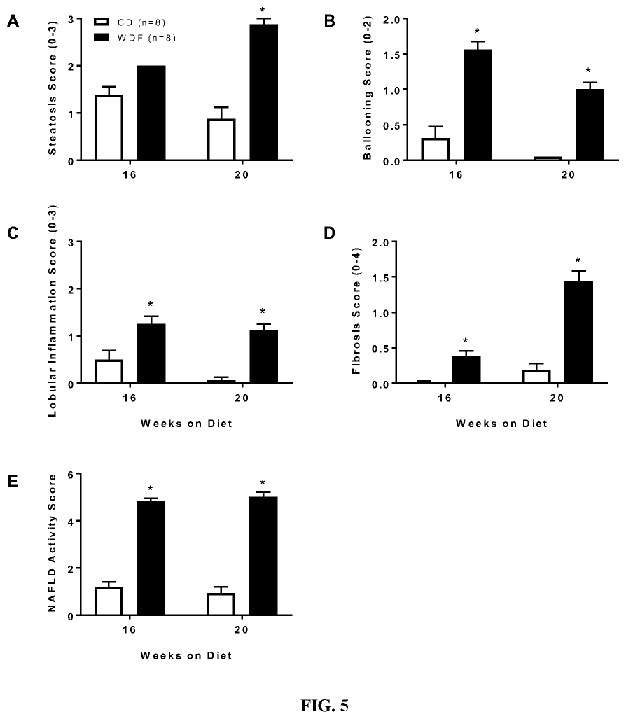

When sections were assessed for NASH activity scores, the livers from WDF fed MS-NASH mice exhibited significantly higher scores for steatosis (FIG. 5A), hepatocellular ballooning (FIG. 5B), lobular inflammation (FIG. 5C) and fibrosis (FIG. 5D) comparing to the corresponding livers from the CD fed mice. In looking at a composite NAFLD activity score, the livers from WDF fed mice demonstrated significantly more pathological findings when compared to the livers from CD fed mice (FIG. 5E).

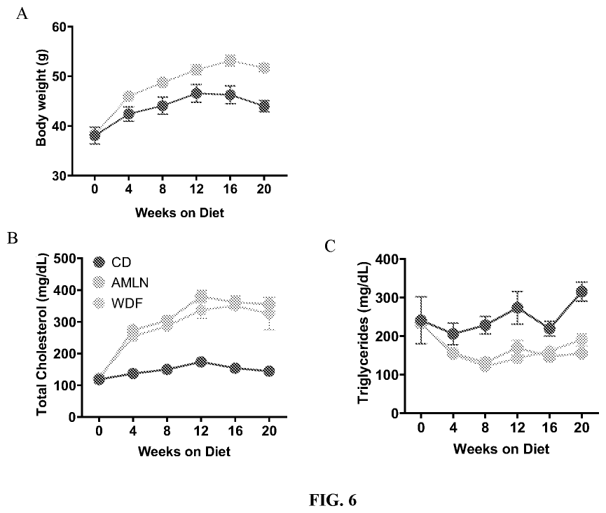

The inventors also tested a high-fat high-fructose diet in which fructose is formulated into the diet instead of provided in drinking water. As shown in FIGS. 6A-6C, the MS-NASH mice fed with high-fat (40% kcal fat) and high-fructose (20% fructose) diet (AMLN diet) showed a significantly greater increase in body weight (FIG. 6A). Blood cholesterol levels were comparably higher in the AMLN and WDF group than CD controls after 4 weeks on diet and the levels were consistently higher in the AMLN and WDF group throughout the diet induction period (FIG. 6B). The triglyceride levels were slightly lower (FIG. 6C) in the AMLN and WDF group than CD controls.

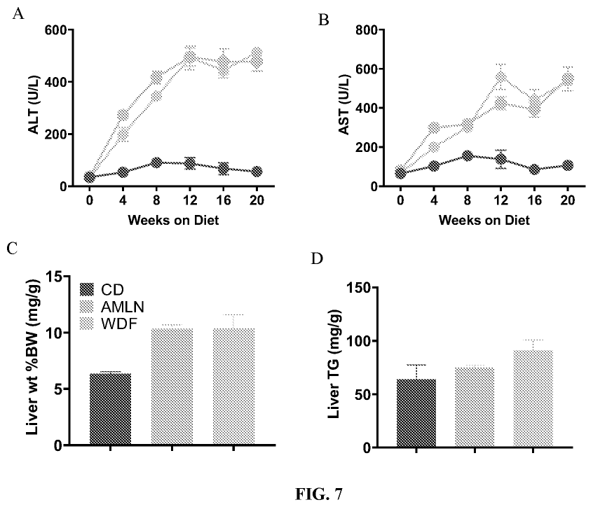

Metabolic stress on the livers of the MS-NASH mice fed with AMLN diet caused significant elevation in the liver enzymes compared to that in control diet (CD) fed mice to the similar level as in the mice fed with WDF diet, with evidence of almost 6 and 4-fold higher in alanine aminotransferase (ALT) (FIG. 7A) and aspartate transaminase (AST) (FIG. 7B) levels respectively. Liver weight was significantly higher in the AMLN and WDF diet group than CD group (FIG. 7C). Liver TG contents measured from mice fed with AMLN diet were comparable to that in the mice fed with CD (FIG. 7D).

### Example 3

This example shows dose effects of CCl₄ in MS-NASH mice fed western diet supplemented with fructose (WDF).

The study aimed to 1) confirm the characterization of MS-NASH mice fed WDF (40% kCal fat, 43% kCal carbohydrate, 17% kCal protein, D12079B, Research Diets, New Brunswick, N.J.) to induce liver phenotypes; and 2) examine the dose effect of CCl₄ (diluted in olive oil, Sigma Aldrich) injected intraperitoneally (IP) twice a week to shorten the induction time and to enhance liver fibrosis.

High dose CCl₄ (0.2 mL/kg), twice weekly for 3 weeks

After 8 weeks on control diet (CD), MS-NASH mice were divided into: 1) CD (n=8): continued on CD for the rest of 11 weeks; 2) WDF (n=8); and 3) WDF+CCl₄ (n=6): switched to WDF for the rest of 11 weeks to induce liver phenotypes; after 8 weeks on WDF, vehicle or CCl₄ was injected IP twice weekly for 3 weeks, respectively.

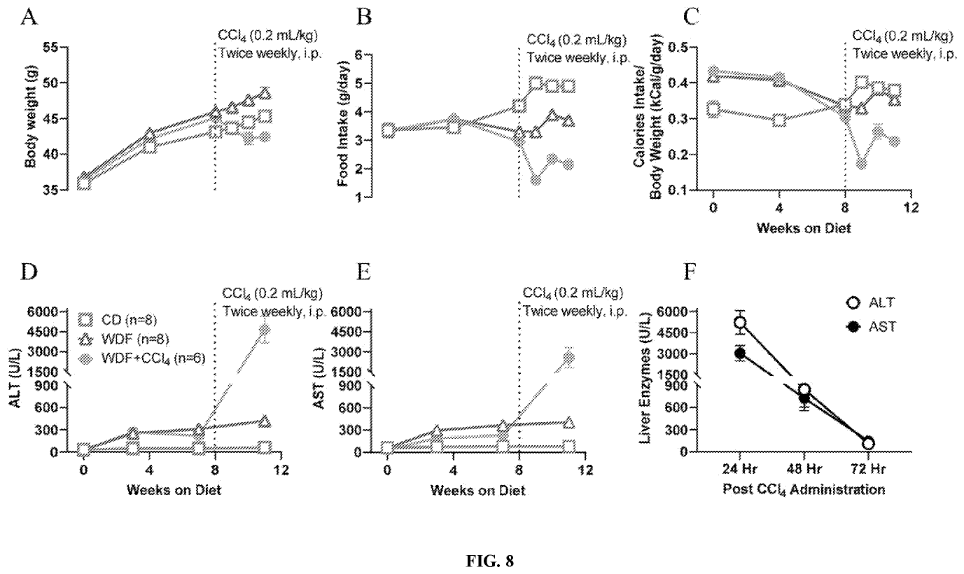

In MS-NASH mice, the present data confirmed that compared to the control diet (CD), WDF enhanced the obesity phenotype (FIG. 8A) with reduction in food (FIG. 8B), but not caloric intake (FIG. 8C), however, it significantly elevated serum ALT (FIG. 8D) and AST (FIG. 8E).

To establish the proper dose of CCl₄ that can accelerate disease progression and enhance liver fibrosis without significant toxic impact on MS-NASH mice, a dose of CCl₄ at 0.2 mL/kg twice weekly was selected, which was a relatively low dose compared to those reported in many studies to induce liver fibrosis in normal rodents without steatosis. Compared to MS-NASH mice on CD or WDF without CCl₄, administration of CCl₄ significantly reduced body weight (FIG. 8A), food (FIG. 8B) and caloric (FIG. 8C) intake, as well as dramatically elevated ALT (FIG. 8D) and AST (FIG. 8E) measured—24 hours after the last dose of CCl₄. The acute response of ASL and ALT to a single dose of CCl₄ at 0.2 mL/kg in a separate experiment showed a similar elevation at 24 hours, but quickly diminished on day 2 and 3 (FIG. 8F).

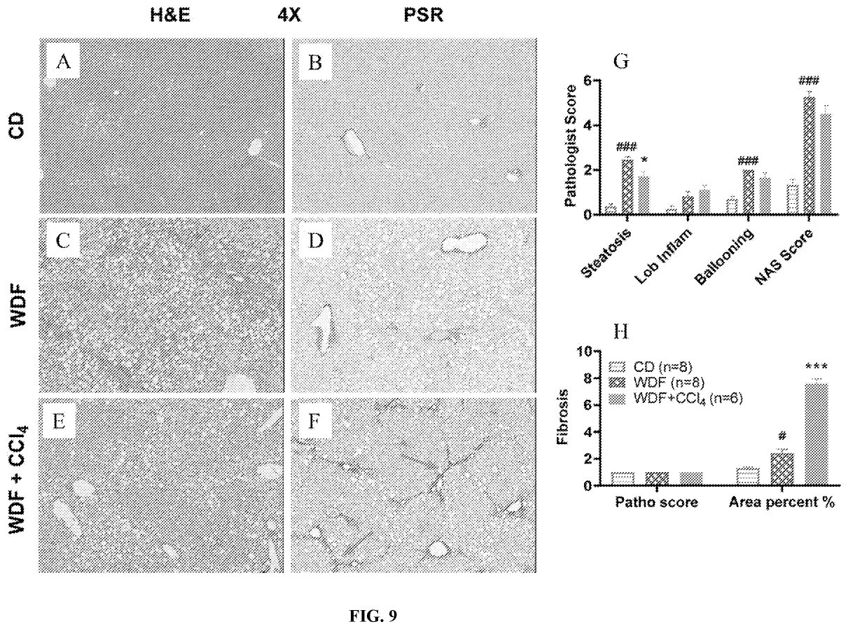

The representative histopathology images showed relatively normal liver in MS-NASH mice on CD (FIGS. 9A and 9B), but a typical NAFLD/NASH pathology in MS-NASH mice on WDF with significantly increased macrovesicular fatty accumulation and microvesicular hepatocyte ballooning (FIGS. 9C and 9D). Although FIGS. 9E and 9F showed that CCl₄ administration in MS-NASH mice on WDF aggravated liver injury and centrilobular fibrosis, pathology scores evaluated by the pathologist failed to detect such enhanced pathology in steatosis, inflammation, ballooning and overall NAS scores from H&E images (FIG. 9G), nor the fibrosis score from PSR images (FIG. 911). However, a quantitative measurement of fibrotic area by computer analysis software (Halo) from PSR images showed a significantly greater fibrosis area in the CCl₄ (˜8%) than CD or WDF (˜2%) group (FIG. 911).

Low dose CCl₄ (0.08 mL/kg), twice weekly for 8 weeks

To further reduce the toxicity of CCL, a separate experiment was performed with the dose of CCl₄ reduced to 0.08 mL/kg twice weekly in MS-NASH mice on WDF. After 8 weeks on CD, MS-NASH mice were switched to WDF for 16 weeks to induce liver phenotypes, which were divided at 8 weeks after WDF into 2 groups: 1) WDF (n=4); and 2) WDF+CCl₄ (n=11).

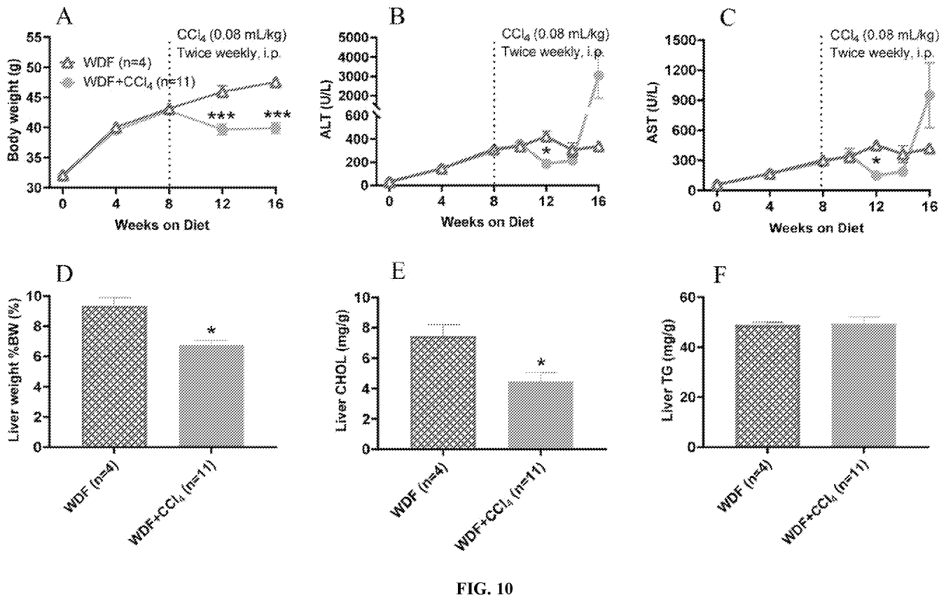

Compared to the mice without CCL, low dose CCl₄ reduced body weight (FIG. 10A), while the elevation of serum ALT (FIG. 10B) and AST (FIG. 10C) was not as dramatic compared to those with high dose CCl₄ measured—24 hours after the last dose of CCl₄ at the end of the experiment. When in-life monitoring of serum ALT and AST was performed 3 days after CCl₄ dosing to minimize influence from acute raise of enzyme levels shown in the high dose experiment, serum ALT and AST levels on week 12 and 14 in the CCl₄ group was significantly lower compared with the mice on WDF only, yet it was still higher than their own baseline before WDF feeding. The liver weight (FIG. 10D) and contents of cholesterol (FIG. 10E), but not triglycerides (FIG. 10F) were significantly reduced by CCl₄.

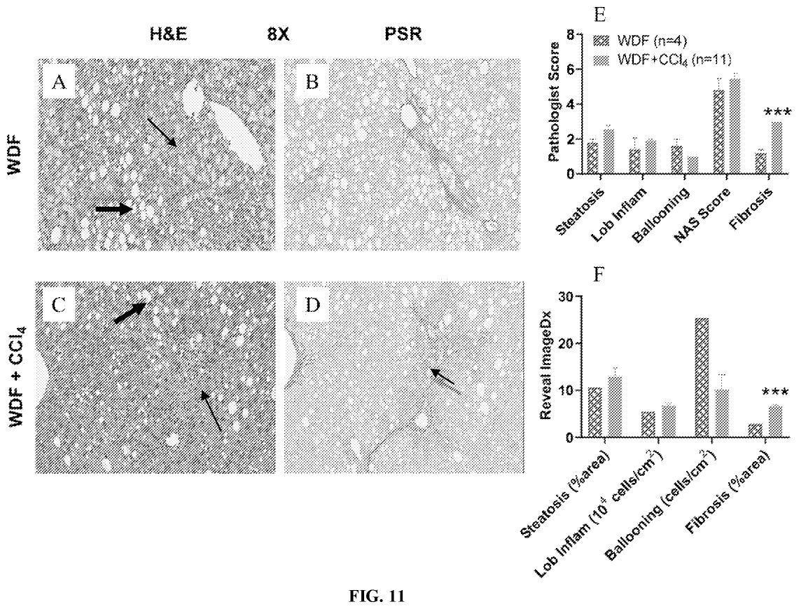

MS—NASH mice on WDF without CCl₄ showed significant steatosis (FIG. 11A) and moderate fibrosis (FIG. 11B). However, MS-NASH mice on WDF treated with CCl₄ presented persisting hepatosteatosis and hepatocyte ballooning degeneration in H&E stained images (FIG. 11C), as well as typical perisinusoidal and periportal fibrosis, along with enhanced bridging fibrosis in PSR stained images (FIG. 11D). The NAS and fibrosis scores evaluated by the pathologist (FIG. 11E) showed significantly aggravated liver fibrosis with little influence on other aspects of liver pathology by low dose CCl₄. Similar to the pathology score analysis, an independent computerized quantitative analysis by Reveal ImageDx also showed larger fibrosis area in mice with CCl₄ compared to those without, but no significant difference in the steatosis area and inflammatory cell infiltration and degenerated liver cell counts between the 2 groups (FIG. 11F).

### Example 4

This example illustrates therapeutic effects of obeticholic acid (OCA) in MS-NASH or C57B1/6 mice on WDF treated low dose CCl₄ (0.08 mL/kg) twice weekly for 8-weeks.

After 8 weeks on CD, MS-NASH mice were fed WDF for 16 weeks to induce liver phenotypes. After 8 weeks on WDF, the animals were injected IP with low dose CCl₄ (0.08 mL/kg) twice weekly and divided into vehicle (n=11) and OCA (n=10) groups for an additional 8 weeks during which, vehicle (1% methylcellulose, Sigma Aldrich) or OCA (Toronto Research Chemicals, New York, ON, Canada, 30 mg/kg) was administrated orally once daily. C57B1/6 mice were compared with the same protocol in vehicle (n=9) or OCA (n=9) groups.

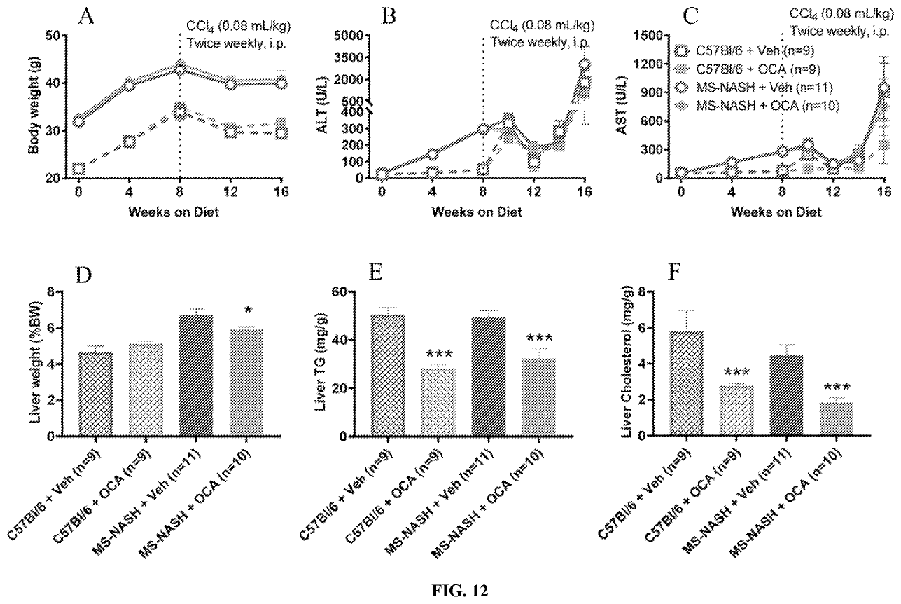

Compared to the vehicle groups, OCA had no significant effect on body weight (FIG. 12A) and serum ALT level (FIG. 12B) in both MS-NASH and C57B1/6 mice, but lowered AST only in C57B1/6 in mice (FIG. 12C). However, OCA significantly reduced liver contents of triglycerides (FIG. 12E) and cholesterol (FIG. 12F) in both MS-NASH and C57B1/6 mice, and reduced liver weight only in MS-NASH mice (FIG. 12D).

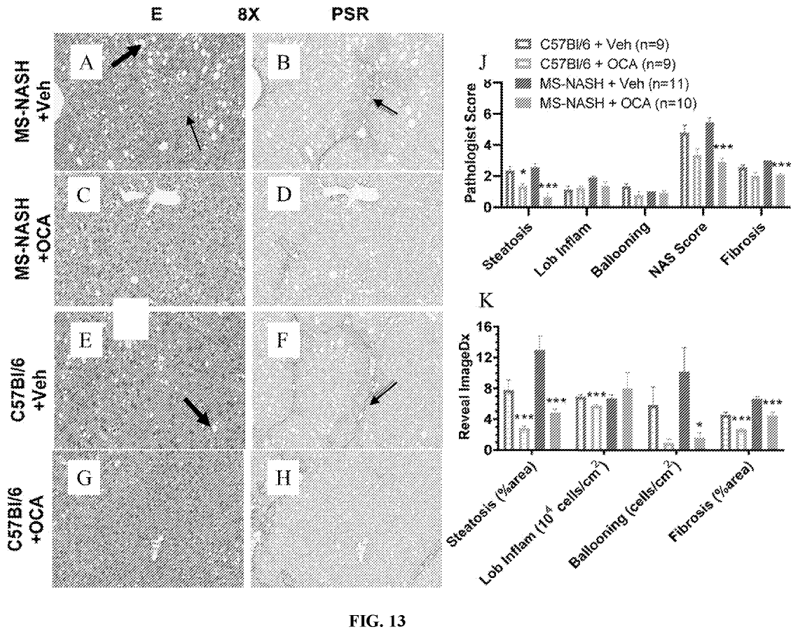

Histopathology images of OCA treated mice (FIGS. 13C, 13D, 13G and 13H) showed less lipid vacuoles and alleviated bridging fibrosis compared to the vehicle treated mice (FIGS. 13A, 13B, 13E, and 13F). These observations were confirmed by both pathologist scoring (FIG. 13J) and computerized quantification (FIG. 13K). Steatosis score by pathologist and percent area by computer quantification were significantly reduced by OCA treatment in both MS-NASH and C57B1/6 mice; quantitative infiltrated inflammatory cell counts in C57B1/6 mice and degenerated ballooning hepatocyte counts in MS-NASH mice were significantly reduced by OCA treatment; NAS and fibrosis scores were significantly reduced in MS-NASH mice by OCA treatment; and percentage fibrosis area was significantly reduced by OCA treatment in both MS-NASH and C57B1/6 mice. Both pathology score and quantitative analysis showed lower NAS and fibrosis scores in C57B1/6 compared to MS-NASH mice, indicating that C57B1/6 mice may require longer NASH induction time and have less hepatocyte ballooning degeneration.

### Example 5

This example illustrates survival rate in MS-NASH and C57B1/6 mice on WDF and treated high and low dose of CCL₄.

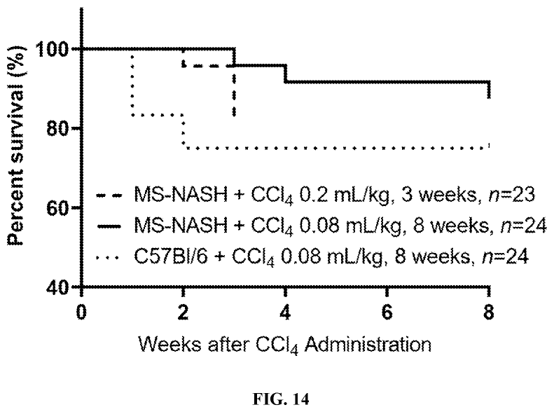

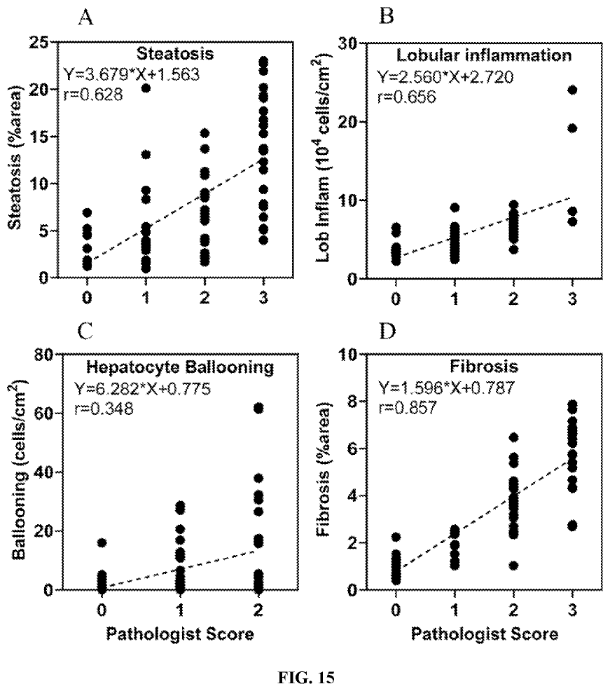

The majority of the mortality occurred in the first 3 weeks of CCl₄ administration in both MS-NASH and C57B1/6 mice. High dose CCl₄ caused death in—20% MS-NASH mice within the first 3 weeks, leading to early termination of the first experiment (FIG. 14). The survival rate in MS-NASH mice under lower dose CCl₄ surpassed those under high dose CCl₄ in the first 3 weeks and reached 87.5% at the end of entire 8-week experimental duration. The survival rate tended to be lower in 57B1/6 than MS-NASH mice with low dose CCl₄. However, this trend was not statistically significant among all the groups.

### Example 6

This example illustrates correlation of imagining analysis between the pathology score and computerized quantification.

A simple linear correlation analysis was performed on 4 aspects of histology readouts between pathologist scoring and quantitative image analysis with ImageDx software. Steatosis (FIG. 15A), lobular inflammation (FIG. 15B), hepatocyte ballooning degeneration (FIG. 15C) and fibrosis (FIG. 15D) scores all showed significant correlations between the 2 independent analyses.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for producing a non-human animal model of non-alcoholic fatty liver disease (NAFLD), the method comprising
(a) obtaining a MS-NASH mouse of a young age; and
(b) feeding the MS-NASH mouse with a diet of high-fat and high fructose and administering to the MS-NASH mouse CCl₄ for a period of time.

2. The method of claim 1, wherein the CCl₄ is administered at about 0.08-0.2 ml per kg body weight of the MS-NASH mouse.

3. The method of claim 1, wherein the CCl₄ is administered via intraperitoneal injection.

4. The method of claim 1, wherein the CCl₄ is administered twice or three times a week.

5. The method of claim 1, wherein the diet comprises 40% kcal fat and 20% kcal fructose.

6. The method of claim 1, wherein the diet comprises 40% kcal fat and 5% fructose in drinking water.

7. The method of claim 1, wherein the NAFLD is steatosis, non-alcoholic steatohepatitis (NASH), cirrhosis or liver cancer.

8. The method of claim 1, wherein the young age is about 8-week old.

9. The method of claim 1, wherein the period of time is 4 weeks, 8 weeks, 12 weeks, 16 weeks or 20 weeks.

10. (canceled)

11. (canceled)

12. (canceled)

13. (canceled)

14. (canceled)

15. (canceled)

16. (canceled)

17. (canceled)

18. (canceled)

19. The method of claim 1, further comprising:
(c) administering a candidate agent to the non-human animal model; and
(d) evaluating an ameliorative effect on the NAFLD.

20. The method of claim 1, further comprising:
(c) administering a medicament to the non-human animal model; and
(d) evaluating an ameliorative effect on the NAFLD.

21. The method of claim 2, wherein the CCl₄ is administered at about 0.08 ml per kg body weight of the MS-NASH mouse.

22. The method of claim 1, wherein the diet comprises 40% kcal fat and 22% kcal fructose.
